(19) United States
(12) Patent Application Publication
(10) Pub. No.: US 2026/0248152 A1
(43) Pub. Date: Aug. 27, 2026

(54) Popping Crystal Compositions

(71) Applicant: HALEON US HOLDINGS LLC, Wilmington, DE (US)

(72) Inventors: Kamala P. Arango, Warren, NJ (US); Kevin D. JORDAN, Warren, NJ (US); Clayton D. GIGELOW, Warren, NJ (US); Charles POTTER, Warren, NJ (US); Edmund Peter MAZIARZ, Warren, NJ (US)

(21) Appl. No.: 19/159,386

(22) PCT Filed: Feb. 26, 2024
(86) PCT No.: PCT/US2024/017215

§ 371 Date: Aug. 25, 2025

Related U.S. Application Data
(60) Provisional application No. 63/487,461, filed on Feb. 28, 2023.

(51) Int. Cl. A23G 3/54; A23G 3/36; A23G 3/42; A61K 9/00; A61K 9/16; A61K 31/375; A61K 31/4415; A61K 31/519; A61K 31/593; A61K 31/714; A61K 33/30; A61K 33/32

(52) U.S. Cl. CPC A23G 3/545; A23G 3/368; A23G 3/42; A61K 9/0056; A61K 9/1611; A61K 9/1617; A61K 9/1623; A61K 9/1652; A61K 31/375; A61K 31/4415; A61K 31/519; A61K 31/593; A61K 31/714; A61K 33/30; A61K 33/32

(57) ABSTRACT

A composition comprising gasified crystals having a particle size of 0.2 mm to 0.85 mm, wherein a 250 mg sample of the gasified crystals generates a cumulative popping signal of up to 0.6 Parms*sec over 10 minutes of dissolution by water.

### TECHNICAL FIELD

The invention is generally related to nutritional supplement compositions, and, more specifically, to nutritional supplement compositions having gasified, popping crystals.

### BACKGROUND

Popping crystals are known in the confectionary industry and are often sold in different flavors as a novelty candy. The popping crystals are typically made of sugar crystals having a pressurized gas contained in cavities within the sugar crystals. As the sugar crystals dissolve or shatter in a user's mouth, the pressurized gas is released, producing is a fun “popping” sensation and sound in a user's mouth. While the popping sensation and sound are enjoyable and fun, this confectionary form is almost exclusively used as a candy, and not as a nutritional, wellness, or pharmaceutical dosage form. There are many reasons for this. For instance, the average particle size of a popping crystal is quite large, being >1 mm in diameter and often being as large as 4 mm in diameter. Additionally, the cavities in these sugar crystals hold a relatively large amount of pressurized gas, which make the popping sensation and sound very noticeable for an extended amount of time, often lasting for a minute or more. Such intense properties, while fun as a novelty candy, are not amenable as a dosage form for nutritional, wellness, or pharmaceutical products that are taken frequently. Moreover, the large particle sizes result in poor composition uniformity when blended with vitamins, minerals, active pharmaceutical ingredients (“API's), sweeteners, and other excipients.

Accordingly, there is a need for popping crystal-containing dosage forms having improved physical properties that amenable for nutritional, wellness, or pharmaceutical products that are taken frequently.

### SUMMARY

In one aspect, a composition comprises gasified crystals having a particle size of 0.2 mm to 0.85 mm, wherein a 250 mg sample of the gasified crystals generates a cumulative popping signal of up to 0.6 Paᵣₘₛ*sec over 10 minutes of dissolution by water. In some embodiments, the cumulative popping signal is between 0.2 to 0.6 Paᵣₘₛ*sec.

In some cases, the gasified crystals comprise a sugar; and pressurized gas entrapped in cavities within the gasified crystals. The sugar comprises glucose, fructose, sucrose, lactose, maltose, corn syrup, glucose syrup, or any combination thereof. In some instances, the sugar comprises sucrose and glucose syrup. The gasified crystals can further comprise an acidulant. In some cases, the acidulant is malic acid.

In some embodiments, the gas comprises carbon dioxide, nitrogen, air, a noble gas, or any combination thereof. The pressurized gas can have a pressure greater than 1 atmosphere in some instances.

In some cases, the gasified crystals have a particle size of 0.5 to 0.7 mm.

In some embodiments, the composition further comprises one or more vitamins, minerals, biological actives, active pharmaceutical ingredients, or any combination thereof.

Exemplary vitamins can comprise vitamin A, a vitamin B (including B3, B6, B9, B12, and biotin), vitamin C, vitamin D, vitamin E, vitamin K, thiamin, riboflavin, niacin, folate, pantothenic acid, pharmaceutically acceptable salts thereof, or combinations thereof. Exemplary minerals can comprise iron, phosphorus, iodine, copper, chromium, molybdenum, chloride, sodium, magnesium, calcium, zinc, selenium, manganese, potassium, choline, silicon, pharmaceutically acceptable salts thereof, or any combination thereof. Exemplary biological actives can comprise lycopene, lutein, L-threonine, coenzyme Q-10, phytosterols, hyaluronic acid, cognizing citicoline, icotinamide, Andrographis, or any combination thereof. Exemplary active pharmaceutical ingredient can comprise a pain or inflammation reducer, an antihistamine, a decongestant, a cough suppressant, a biological active, a sleep aid, a stress-reducing aid, an energy and cognition aid, an Ayurveda supplement, an immunity supplement, a digestive aid, a joint supplement, a cannabinoid, a systemic natural, a nutritional agent, pharmaceutically acceptable salts thereof, or combinations thereof.

In some embodiments, the composition further comprises a sweetener. In some cases, the sweetener comprises glucose, fructose, sucrose, lactose, maltose, corn syrup, or any combination thereof.

The composition can further comprise a diluent, a flavorant, a colorant, a preservative, or any combination thereof.

In another aspect, a composition comprises gasified crystals having a particle size of 0.25 to 0.85 mm, the gasified crystals comprising sucrose, glucose syrup, and a pressurized gas entrapped in cavities within the gasified crystals; wherein a 250 mg sample of the gasified crystals generates a popping signal of 0.2 to 0.6 Paᵣₘₛ*sec over 10 minutes of dissolution by water. The gas is carbon dioxide pressurized at greater than 1 atmosphere in some cases. The composition can further comprise one or more vitamins, minerals, biological actives, active pharmaceutical ingredients, or any combination thereof in some instances. In some embodiments, the composition further comprises an acidulent. An exemplary acidulent is malic acid.

### BRIEF DESCRIPTION OF THE DRAWINGS

FIG. 1 is a block diagram of a method of preparing gasified crystals;

FIG. 2 is a perspective view of an acoustic apparatus;

FIG. 3 is a cross-sectional view of the acoustic apparatus of FIG. 2;

FIG. 4 is a perspective view of a sample wetting cup;

FIG. 5 is a side view of the sample wetting cup of FIG. 4;

FIG. 6 is a top view of the sample wetting cup of FIG. 4;

FIG. 7 is a bottom view of the sample wetting cup of FIG. 4;

FIG. 8 is a cross-sectional side view of the sample wetting cup of FIG. 5; and

FIG. 9 is a cross-section side view of the sample wetting cup of FIG. 5 connected to a fluid injection nozzle;

FIG. 10 is photograph of the sample wetting cup of FIG. 4 having a sample prior to addition of a wetting fluid;

FIG. 11 is a photograph of the sample wetting cup of FIG. 10 after dissolution of the sample by water;

FIG. 12 is a photograph of the sample wetting cup of FIG. 10 having a drain hole;

FIG. 13 is a graph showing blending uniformity of larger gasified crystal-containing compositions;

FIG. 14 is a graph showing blending uniformity of smaller gasified crystal-containing compositions;

FIG. 15 is a plot showing cumulative popping signals of gasified crystals having different particle size ranges; and

FIG. 16 is a plot showing comparative data of the cumulative popping signals of gasified crystals having different particle size ranges and commercial novelty candy.

### DETAILED DESCRIPTION

Embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10.

The unit of “percentage by weight” is interchangeably represented as “% by weight” or “wt. %” unless expressly stated otherwise.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

A composition suitable as a nutritional, wellness, or pharmaceutical supplement is described herein. In one aspect, the composition is an edible composition comprising gasified crystals. As described in more details in EXAMPLE 1, a consumer preference study found that while consumers enjoyed the popping sensation and sounds produced by gasified crystals found in novelty candy, they were not likely to regularly consume such products as part of a nutritional, wellness, or pharmaceutical regime, because the intensity and duration of the popping was too intense and long. However, additional studies found that consumers favorably responded to reduced popping intensity and duration. Specifically, it was discovered that the most favorable compositions were those comprising gasified crystals having a specific particle distribution size of 0.2 to 0.85 mm. Particularly, as described in more detail herein, it was found that gasified crystals having a particle size of 0.2 to 0.85 mm, where a 250 mg sample of the gasified crystals generates a cumulative popping signal of up to 0.6 Paᵣₘₛ*sec over 10 minutes of dissolution by water were the most preferred.

Moreover, in addition to having the desired cumulative popping signal, the gasified crystal particle size range of 0.2 to 0.85 mm was discovered to have superior blending uniformity properties. As described in EXAMPLE 2 in more detail, compositions have larger gasified crystal sizes (e.g., >0.85 mm, such as between 0.85 mm to 4 mm), other ingredients in the composition readily separate out from the gasified crystals, resulting in poor blend uniformity.

These gasified crystals can further comprise one or more vitamins, minerals, biological actives, API's, or any combination thereof; and additional excipients such as an acidulant, an acidifier, a flavorant, a diluent, a colorant, a preservative, or any combination thereof. Compositions, methods of making the compositions, and devices and analytical methods for determining popping intensity and duration of gasified crystals are described herein.

### I. Compositions

In an aspect, a composition described herein is an edible composition comprising gasified crystals. In some embodiments, the gasified crystals in the composition have a particle size of 0.25 to 0.85 mm. In some cases, the gasified crystals have a particle size of 0.25 to 0.8 mm, 0.25 to 0.75 mm, 0.25 to 0.7 mm, 0.25 to 0.65 mm, 0.25 to 0.6 mm, 0.25 to 0.55 mm, 0.25 to 0.5 mm, 0.25 to 0.45 mm, 0.25 to 0.4 mm, 0.25 to 0.35 mm, 0.25 to 0.3 mm, 0.3 to 0.85 mm, 0.35 to 0.85 mm, 0.4 to 0.85 mm, 0.45 to 0.85 mm, 0.5 to 0.85 mm, 0.55 to 0.85 mm, 0.6 to 0.85 mm, 0.65 to 0.85 mm, 0.7 to 0.85 mm, 0.8 to 0.85 mm, 0.3 to 0.8 mm, 0.35 to 0.75 mm, 0.4 to 0.7 mm, 0.45 to 0.65 mm, 0.5 to 0.65 mm, 0.5 to 0.6 mm, 0.55 to 0.65 mm, 0.5 to 0.66 mm, 0.5 to 0.7 mm, 0.45 to 0.75 mm, 0.45 to 0.7 mm, or 0.45 to 0.65 mm.

In some embodiments, the gasified crystals are present in the composition in an amount of 5% to 60%, 5% to 55%, 5% to 50%, 5% to 45%, 5% to 40%, 5% to 35%, 5% to 30%, 5% to 25%, 5% to 20%, 5% to 15%, 5% to 10%, 10% to 60%, 15% to 60%, 20% to 60%, 25% to 60%, 30% to 60%, 35% to 60%, 40% to 60%, 45% to 60%, 50% to 60%, 55% to 60%, 10% to 55%, 15% to 50%, 20% to 45%, 25% to 40%, 30% to 35%, 5%, 10%, 15%, 20%, 25%, 30%, 35%, 40%, 45%, 50%, 55% or 60% by weight of the composition.

In some instances, the gasified crystals comprise a sugar and pressurized gas entrapped in cavities within the gasified crystals. The term “sugar” includes a monosaccharide, a disaccharide, a polysaccharide, sugar alcohols, or any combination thereof. A monosaccharide described herein can comprise fructose, glucose, or any combination thereof. A disaccharide described herein can comprise sucrose, lactose, maltose, or any combination thereof. A polysaccharide described herein can comprise a syrup, such as a glucose syrup, a corn syrup, a high fructose corn syrup, an agave syrup, a high maltose syrup, a rice syrup (such as brown rice syrup), or any combination thereof. A sugar alcohol described herein can comprise erythritol, sorbitol, xylitol, mannitol, or any combination thereof.

In some cases, the sugar comprises glucose, fructose, sucrose, lactose, maltose, glucose syrup, corn syrup, or any combination thereof. In some instances, the sugar comprises sucrose and glucose syrup.

The amount of sugar present in the gasified crystals can be any amount not inconsistent with the objectives of this disclosure. For example, in some cases the gasified crystals can comprise 90-99.9% sugar. In some instances, the gasified crystals comprise 91-99.9%, 92-99.9%, 93-99.9%, 94-99.9%, 95-99.9%, 96-99.9%, 97-99.9%, 98-99.9%, 99-99.9%, 91%, 92%, 93%, 94%, 95%, 96%, 97%, 98%, 99%, or >99% but less than 100% by weight of sugar.

The pressurized gas entrapped in cavities within the gasified crystals comprises an inert gas. The term “inert” means a gas that does not chemically react with the materials forming the gasified crystals (such as the sugar), or any vitamin, mineral, or API present in the composition, whether the vitamins, minerals, or API are present in the gasified crystal material itself or present as a blend with the gasified crystals. Exemplary gasses include carbon dioxide, nitrogen, air, a noble gas, or any combination thereof.

The term “pressurized” refers to a pressure greater than 1 atmosphere in some embodiments. In some cases, the term “pressurized” refers to a pressure that is greater than an ambient pressure outside the cavities within the gasified crystals.

The pressurized gas can be released from the cavities in the gasified crystals to produce the popping sensation and sound experienced by a user, such as upon dissolution of the gasified crystals in water, contact of the gasified crystals with saliva in a consumer's mouth, and/or when the gasified crystals are shattered upon chewing. As described in more detail in Sections III and IV herein, a cumulative popping signal can be measured for the gasified crystals during dissolution of the gasified crystals by water. Specifically, a cumulative popping signal can be determined for the desired popping intensity, such as for gasified crystals with a defined particle size range that consumers identified as favorable in a consumer study discussed in EXAMPLE 1. In some embodiments, a 250 mg sample of the gasified crystals described herein generates a cumulative popping signal of up to 0.6 Paᵣₘₛ*sec over 10 minutes of dissolution by water. In some cases, a 250 mg sample of the gasified crystals generate a cumulative popping signal of up to 0.55 Paᵣₘₛ*sec, up to 0.5 Paᵣₘₛ*sec, up to 0.45 Paᵣₘₛ*sec, up to 0.4 Paᵣₘₛ*sec, up to 0.35 Paᵣₘₛ*sec, up to 0.3 Paᵣₘₛ*sec, up to 0.25 Paᵣₘₛ*sec, up to 0.2 Paᵣₘₛ*sec, between 0.2 to 0.6 Paᵣₘₛ*sec, 0.25 to 0.6 Paᵣₘₛ*sec, 0.3 to 0.6 Paᵣₘₛ*sec, 0.35 to 0.6 Paᵣₘₛ*sec, 0.4 to 0.6 Paᵣₘₛ*sec, or 0.5 to 0.6 Paᵣₘₛ*sec over 10 minutes of dissolution by water.

In some embodiments, the composition can further comprise a sweetener. In some cases, the sweetener is a natural sugar comprising glucose, fructose, sucrose, lactose, maltose, corn syrup, yacon syrup, monk fruit, honey, agave, coconut sugar, date sugar, fruit puree, fruit juices, maple syrup, molasses, or any combination thereof. In some instances, the sweetener can also be an artificial sweetener, such as stevia, erythritol, xylitol, allulose, acesulfame-K, aspartame, neotame, saccharin, sucralose, or any combination thereof. Additionally, it is also contemplated that in some embodiments, a combination of natural sugar and artificial sweeteners can be used. The amount of sweetener present in the composition can be any amount not inconsistent with the objectives of this disclosure. In some instances, the sweetener is present in the composition in an amount of 10% to 80%, 10% to 75%, 10% to 70%, 10% to 65%, 10% to 60%, 10% to 55%, 10% to 50%, 10% to 45%, 10% to 40%, 10% to 45%, 10% to 40%, 10% to 35%, 10% to 30%, 10% to 25%, 10% to 20%, 10% to 15%, 15% to 80%, 20% to 80%, 25% to 80%, 30% to 80%, 35% to 80%, 40% to 80%, 45% to 80%, 50% to 80%, 55% to 80%, 60% to 80%, 65% to 80%, 70% to 80%, 15% to 60%, 20% to 55%, 25% to 50%, 30% to 45%, 10%, 15%, 20%, 25%, 30%, 35%, 40%, 45%, 50%, 55%, 60%, 65%, 70%, 75%, or 80% by weight of the composition.

The composition can further comprise one or more vitamins, minerals, biological actives, API's, or any combination thereof. In some cases, the composition comprises one or more vitamins, minerals, biological actives, API's, or any combination thereof blended with the gasified crystals. In some instances, the composition comprises a gasified crystal comprising one or more vitamins, minerals, API's, or any combination thereof. In some embodiments, each of the one or more vitamins, minerals, biological actives, and API's can be present in the composition as both blended with the gasified crystal and incorporated in the gasified crystal.

The vitamin, mineral, or biological active comprise vitamin A, a vitamin B (including B3, B6, B9, B12, and biotin), vitamin C, vitamin D, vitamin E, vitamin K, thiamin, riboflavin, niacin, folate, pantothenic acid, iron, phosphorus, iodine, copper, chromium, molybdenum, chloride, sodium, magnesium, calcium, zinc, selenium, manganese, potassium, choline, lycopene, lutein, L-threonine, coenzyme Q-10, phytosterols, hyaluronic acid, cognizing citicoline, silicon, icotinamide, Andrographis, pharmaceutically acceptable salts thereof, or combinations thereof.

In some embodiments, the API comprises a pain or inflammation reducer, an antihistamine, a decongestant, a cough suppressant, a biological active, a sleep aid, a stress-reducing aid, an energy and cognition aid, an Ayurveda supplement, an immunity supplement, a digestive aid, a joint supplement, a cannabinoid, a systemic natural, a nutritional agent, pharmaceutically acceptable salts thereof, or combinations thereof.

Exemplary pain or inflammation reducer comprises ibuprofen, naproxen, acetaminophen, salicylic acid, acetylsalicylic acid, ketoprofen, dexibuprofen, fenoprofen, dexketoprofen, flurbiprofen, oxaprozin, loxoprofen, diflunisal, etodolac, indomethacin, ketorolac, piroxicam, salsalate, salicylic acid, indomethacin, tolmetin, sulindac, etodolac, ketorolac, diclofenac, aceclofenac, bromfenac, pharmaceutically acceptable salts thereof, or combinations thereof.

The antihistamine comprises cetirizine hydrochloride, levocetirizine hydrochloride, loratadine, desloratadine, fexofenadine hydrochloride, azelastine hydrochloride, olopatadine hydrochloride, brompheniramine maleate, chlorcyclizine hydrochloride, chlorpheniramine maleate, dexbrompheniramine maleate, dexchlorpheniramine maleate, diphenhydramine citrate, diphenhydramine hydrochloride, doxylamine succinate, phenindamine tartrate, pheniramine maleate, pyrilamine maleate, thonzylamine hydrochloride, triprolidine hydrochloride, pharmaceutically acceptable salts thereof, or combinations thereof.

The decongestant or cough suppressant comprises phenylephrine hydrochloride, pseudoephedrine hydrochloride, pseudoephedrine sulfate, phenylephrine bitartrate, dextromethorphan, pholcodine, codeine, benzonatate, pharmaceutically acceptable salts thereof, or combinations thereof.

The sleep aid comprises melatonin, lemon balm, lavender, chamomile, sage, valerian root extract, hops, passionflower extract, or combinations thereof.

The stress-reducing aid comprising sage, Ashwagandha (Withania somnifera), St. John's Wort, GABA (gamma-Aminobutyric acid), or combinations thereof.

The energy and cognition aid comprising green tea, ginseng, caffeine, rhodiola extract, B-vitamins, L-theanine, taurine, pharmaceutically acceptable salts thereof, or combinations thereof.

The Ayurveda supplement comprises amla (Emblica officinalis), bibhitaki (Terminalia bellirica), haritaki (Terminalia chebula)), Brahmi, cumin, licorice root, Gotu kola, Cardamom, or combinations thereof.

The immunity supplement comprises copper, silver, spirulina, astragalus, beta glucan, acerola cherry extract, blood orange extract, elderberry, mushrooms (Lions Mane, Reishi, Shiitake, Cordyceps), pharmaceutically acceptable salts thereof, or any combinations thereof.

The digestive aid comprises a probiotic and combinations thereof; digestive enzymes; soluble fibers including psyllium husk, inulin, fenugreek fiber, wheat dextrin; Cascara sagrada; aloe ferox leaves extract, slippery elm bark extract, or combinations thereof.

The joint supplement comprises collagen, glucosamine, turmeric, methylsulfonylmethane (MSM), pharmaceutically acceptable salts thereof, or combinations thereof.

The cannabinoid comprises cannabidiol, cannabigerol, pharmaceutically acceptable salts thereof, and combinations thereof.

The systemic natural comprises one or more of Boswellia, curcumin, devils claw, ginger, feverfew, bromelain, turmeric, and butterbur.

The nutritional agent comprises black pepper, glycine, valerian root, eucalyptus, menthol, astragalus, bromelain, echinacea, white willow, ivy leaf (Hedera helix), ginger (Zingiber officinale), pharmaceutically acceptable salts thereof, or combinations thereof.

Compositions described herein can further comprise additional excipients, such as an acidulant, a diluent, a flavorant, a colorant, a preservative, or any combination thereof.

Exemplary acidulants include malic acid, citric acid, turmeric acid, lactic acid, tartaric acid, acetic acid, sodium ascorbate, ascorbic acid, succinic acid, or any combination thereof. In some embodiments, the acidulant is malic acid. In some cases, the composition comprises one or more acidulants blended with the gasified crystals. In some instances, the composition comprises a gasified crystal comprising one or more acidulants. In some embodiments, one or more acidulants are present in the composition as both blended with the gasified crystal and incorporated in the gasified crystal.

Exemplary diluents include maltodextrin, starches, cellulosics, sugars, fibers, or any combination thereof. When present the amount of diluent can be any amount not inconsistent with the objectives of this disclosure.

Optionally, a flavorant can be used in the composition. When present, the flavorant can be any flavorant known in the art that is not inconsistent with the objectives of this disclosure. As known by the skilled artisan, the amount of flavorant present will depend on the desired flavor, the specific flavorant being used, and whether the flavorant is an ingredient of the gasified crystal material or is present separately as a blend in the composition. In some cases, the composition comprises a flavorant blended with the gasified crystals. In some embodiments, the composition comprises a gasified crystal comprising a flavorant. In some embodiments, the flavorant is present in the composition as both blended with the gasified crystal and incorporated in the gasified crystal.

Optionally, a colorant can be used in the composition. When present, the colorant can be any colorant known in the art that is not inconsistent with the objectives of this disclosure. As known by the skilled artisan, the amount of colorant present will depend on the desired color, the specific colorant being used, and whether the colorant is an ingredient of the gasified crystal material or is present separately as a blend in the composition. In some cases, the composition comprises a colorant blended with the gasified crystals. In some embodiments, the composition comprises a gasified crystal comprising a colorant. In some embodiments, the colorant is present in the composition as both blended with the gasified crystal and incorporated in the gasified crystal.

In some instances, a preservative is present in the composition. The preservative can be any preservative not inconsistent with the objectives of this disclosure. Exemplary preservatives include sodium benzoate, benzyl alcohol, methyl paraben, propyl paraben, or any combination thereof. The preservative can be present in the composition in any amount not inconsistent with the objectives of this disclosure. In some cases, the composition comprises a preservative blended with the gasified crystals, and in other cases, the composition comprises a gasified crystal comprising a preservative. In some embodiments, the preservative is present in the composition as both blended with the gasified crystal and incorporated in the gasified crystal.

### II. Method of Making Compositions

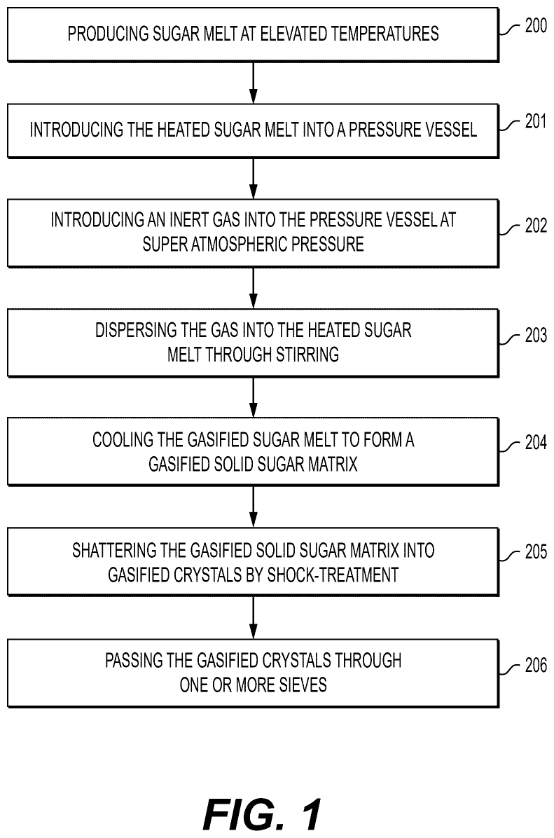

In another aspect, methods of preparing compositions disclosed in Section I herein are described. In some embodiments, gasified crystals can be prepared using methods known in the art. By way of example, U.S. Pat. Nos. 3,985,909, 3,985,910, 4,001,457, and 4,289,794 describe methods for making gasified crystals. As shown in FIG. 1, a method of preparing gasified crystals comprises producing a sugar melt at elevated temperatures 200; introducing the heated sugar melt into a pressure vessel 201; introducing an inert gas into the pressure vessel at super atmospheric pressure 202; dispersing the gas into the heated sugar melt through stirring 203; cooling the gasified sugar melt to form a gasified solid sugar matrix 204; shattering the gasified solid sugar matrix into gasified crystals by shock-treatment 205; and passing the gasified crystals through one or more sieves 206 to obtain the desired particle size range and/or distribution.

Compositions described in Section I can be prepared by blending the gasified crystals with a sweetener, additional excipients, and one or more vitamins, minerals, and/or API's.

### III. Analytical Apparatus

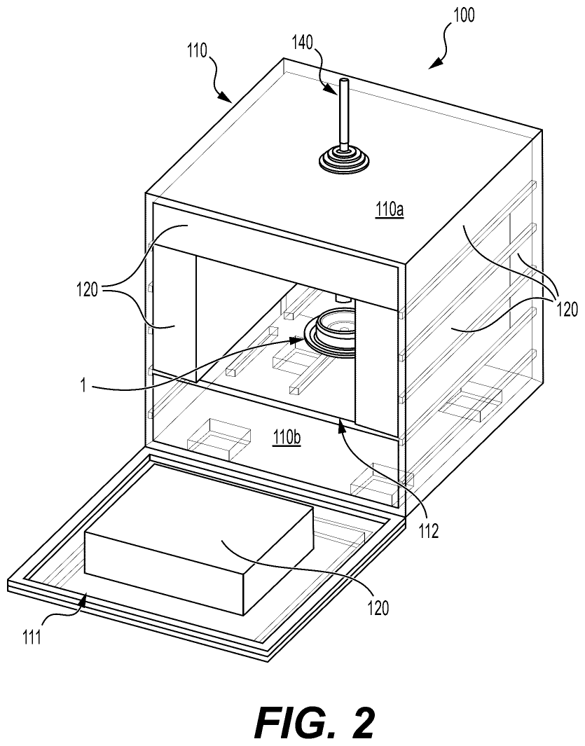

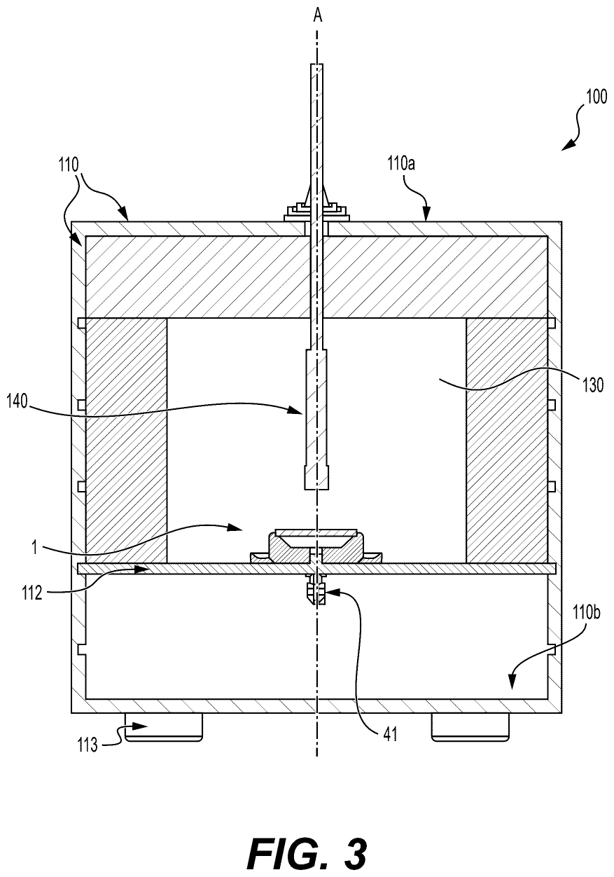

In an embodiment shown in FIGS. 2 and 3, acoustic apparatus 100 comprises an insulated container 110 having a closeable door 111, a sample receiving platform 112, and sound reducing insulation 120 lining the interior-facing surface of each of the walls comprising the insulated container 110 and door 111. Exemplary sound reducing insulation can include acoustic foam with or without sound reducing surface patterns such as “eggshell” patterns, or any other sound reducing insulation that is not inconsistent with the objectives of this disclosure. In the embodiment of FIG. 2, the closeable door 111 is positioned on one of the vertical sides of container 110. However, in other cases, door 111 can be positioned as a lid on the top horizontal wall 110a of container 110. Container 110 can be a cube or rectangular shape having a centrally located interior receiving space 130. A removable wetting cup 1 is positioned on sample receiving platform 112 in the interior receiving space 130. The sample receiving platform 112 has a first receiving hole passing therethrough, upon which the wetting cup 1 is positioned. As discussed in more detail below, wetting cup 1 is positioned such that a fluid receiving hole 40 in the wetting cup 1 is aligned with the first receiving hole.

Vibration dampening feet 113 can optionally be positioned on an outer surface of a bottom horizontal wall 110b of container 110. Feet 113 can be made from any vibration absorbing or dampening material not inconsistent with the objectives of this disclosure, such as a natural or synthetic rubber. Generally, the purpose of the vibration dampening feet 113 is to reduce external vibrations felt by the acoustic apparatus 100. In addition to or in the absence of vibration dampening feet 113, placing the acoustic apparatus 100 on a vibration resistant surface can be used with or in the place of vibration dampening feet 113.

A second receiving hole passes through the top horizontal wall 110a and insulation 120 positioned thereof. A microphone 140 or other measuring device can be positioned in the second receiving hole. In an embodiment shown in FIGS. 2 and 3, microphone 140 passes through the second receiving hole and extends into the interior receiving space 130. In some instances, the first receiving hole, second receiving hole, and fluid receiving hole 40 are approximately aligned along a common axis A, although in other embodiments, the holes are positioned in a configuration other than along axis A. The microphone 140 is positioned above an approximate center of wetting cup 1 in some embodiments. In other cases, the second receiving hole can pass through one of the vertical sidewalls of container 110, and the microphone 140 extends horizontally into the interior receiving space 130.

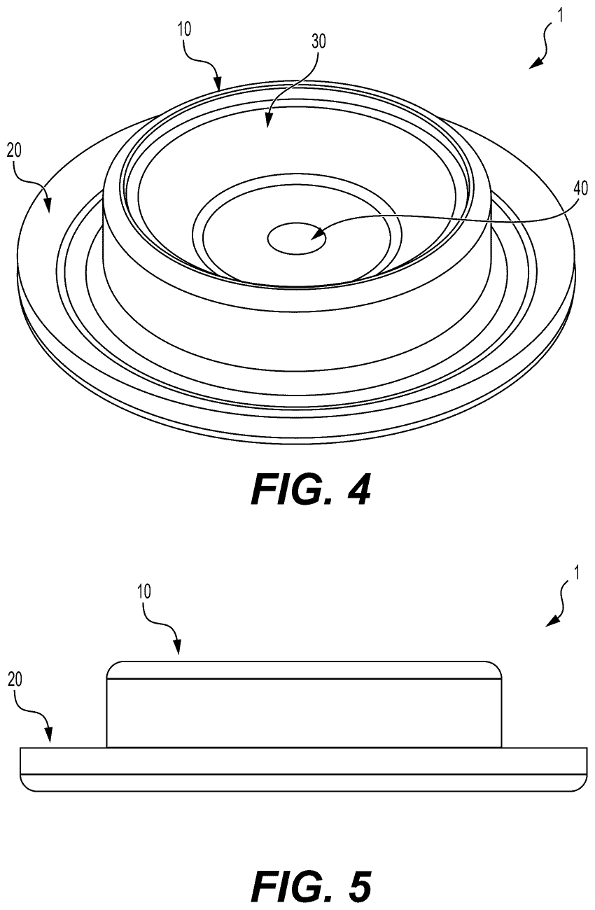

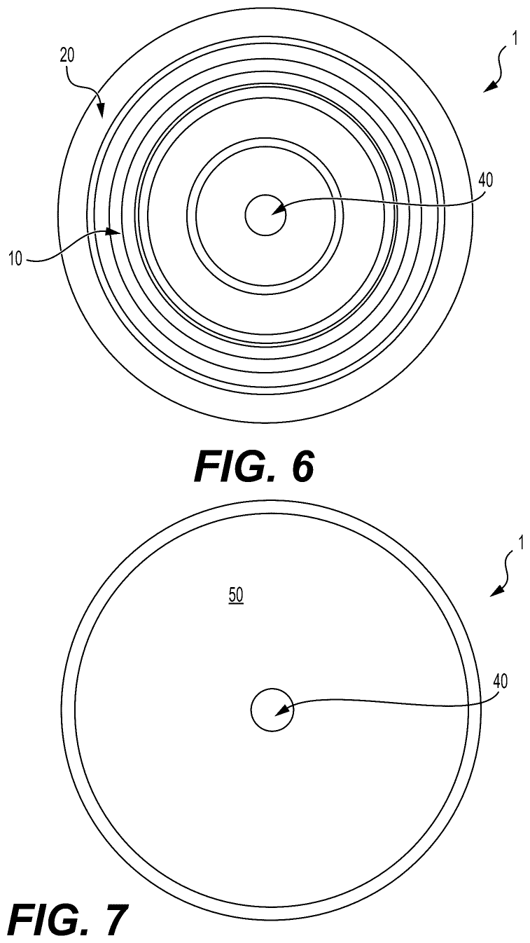

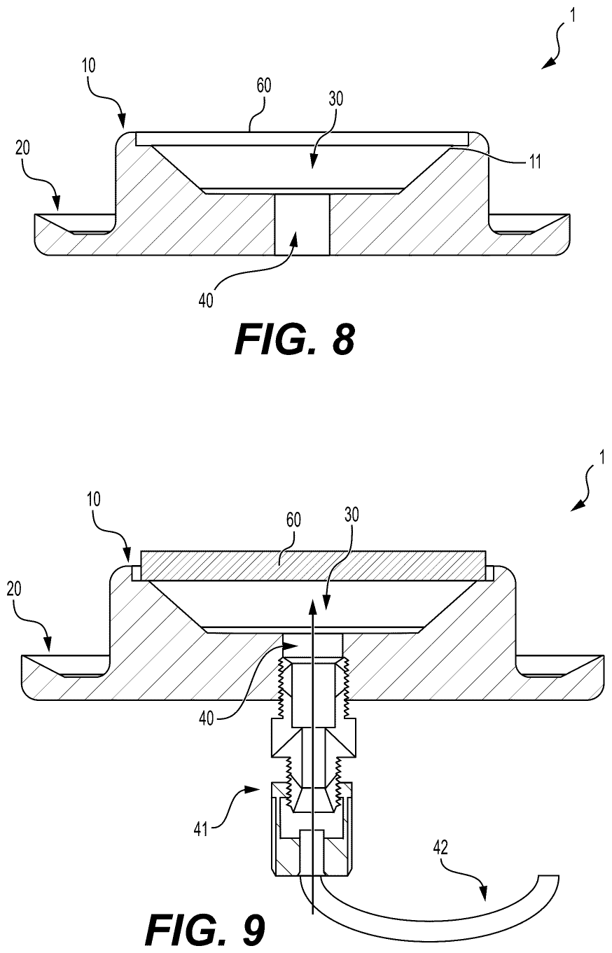

In an aspect shown in FIGS. 4 to 11, wetting cup 1 comprises a fluid receiving bowl 10; a fluid overflow base 20; a fluid receiving space 30 positioned within the fluid receiving bowl 10; and the fluid receiving hole 40. The fluid receiving bowl 10 has a circumferentially extending shelf 11 positioned on an inward facing surface proximate to a rim of the fluid receiving bowl 10. A fritted disc 60 can be positioned on shelf 11 over the fluid receiving bowl 10 to cover fluid receiving space 30. The fritted disc 60 can be made of glass, polymer, or any other material not inconsistent with the objectives of this disclosure. In an embodiment shown in FIG. 8, an outer surface of fritted disc 60 can be level with the rim of the fluid receiving bowl 10. In another embodiment shown in FIG. 9, an outer surface of fritted disc 60 can extend further outward from the rim of the fluid receiving bowl 10. In instances where capillary action is used to carry fluid from the fluid receiving bowl 10 to the outer surface of fritted disc 60 to contact sample 70, the embodiment shown in FIG. 9 is generally used.

In some embodiments, fluid overflow base 20 has a bottom surface 50 that can be placed on the sample receiving platform 112 in the interior receiving space 130 of acoustic apparatus 100. In some instances, bottom surface 50 is flat. A nozzle 41 can be removably attached to the fluid receiving hole 40, such as through a friction fit or screwing mechanism. In other cases, nozzle 41 is integrally formed as part of the fluid overflow base 20, extending outward from the bottom surface 50. As shown for example, in FIG. 9, nozzle 41 can be attached to the fluid receiving hole 40, and a fluid conduit 42, such as a tube, can be attached to an end of nozzle 41. Fluid conduit 42 can connect nozzle 41 with a fluid dispensing device, such as a syringe or other known fluid dispensing device (not shown).

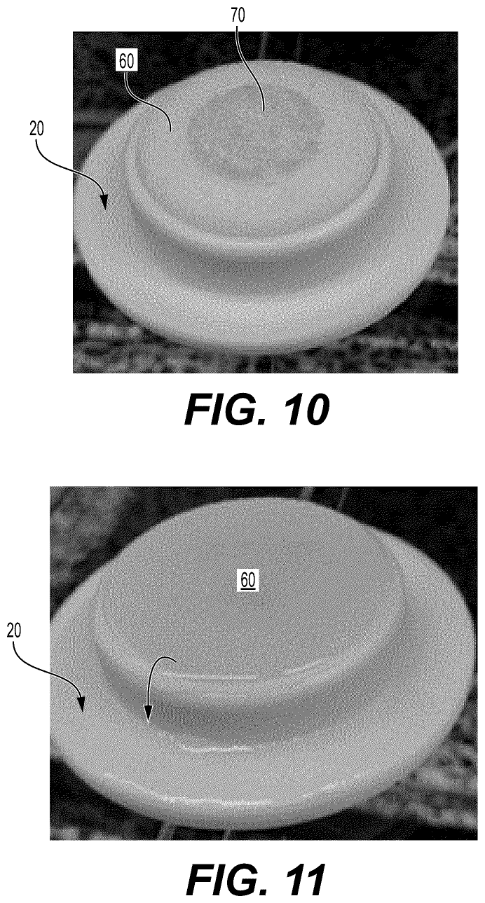

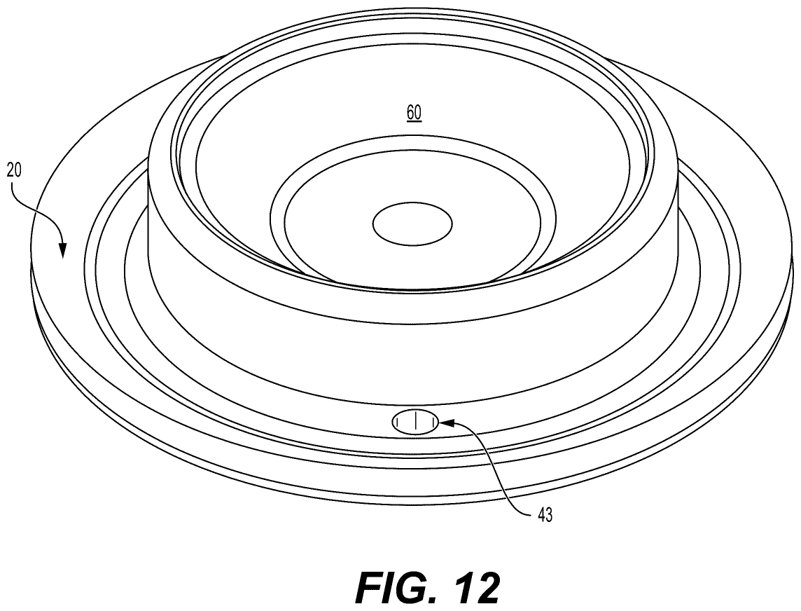

As shown in FIG. 3, wetting cup 1 can be placed on sample receiving platform 112 such that the fluid receiving hole 40 and the first receiving hole are aligned along axis A. A fluid conduit 42 can connect nozzle 41 to a fluid dispensing device (such as a syringe), and a known volume of fluid can be passed through fluid conduit 42 and nozzle 41 to fill the fluid receiving space 30 of the fluid receiving bowl 10. As the fluid rises in the fluid receiving bowl 10, the fluid can contact the fritted disc 60. So long as wetting cup 1 is level, the fluid will evenly contact the bottom surface of the fritted disc 60 and be drawn up through the fritted disc 60 and wet an outer surface of the fritted disc 60 in a uniform manner. Capillary action will then draw the fluid through the fritted disc 60 and uniformly/evenly wet the surface of the fritted disc 60. As shown in FIGS. 10 and 11, a sample 70 can be placed on the outer surface of fritted disc 60. A fluid (such as water or a solvent) can be injected into the fluid receiving space 30 of bowl 10, and when the fluid contacts the bottom of fritted disc 60, the fluid is drawn up into the fritted disc 60 through capillary action and contacts sample 70.

As shown in FIG. 11, the volume of fluid injected into the fluid receiving space 30 can be greater than a volume of the fluid receiving space 30 to ensure the fritted disc 60 is completely saturated and wetted with the fluid. Any excess fluid can spill over the rim of the fluid receiving bowl 10 and be captured by the fluid overflow base 20. In some cases, a fixed volume of fluid can be injected into the fluid receiving space 30. In other cases, a continuous flow of fluid can be injected into the fluid receiving space 30. In instances where a continuous flow of fluid is used, a draining hole 43 can be formed in the fluid overflow base 20 to drain the excess fluid. Similar to the fluid receiving hole 40, the draining hole 43 can be connected to a second nozzle 41 and a second fluid conduit 42 to drain the fluid away from the wetting cup 1.

### IV. Analytical Methodology

In another aspect, acoustic apparatus 100 can be used to measure a popping intensity and/or duration of the gasified crystals upon dissolution in water. Specifically, acoustic apparatus 100 can be used to determine cumulative popping signal over time, which is expressed in units of Pascal RMS seconds (Paᵣₘₛ*sec), where RMS means root mean square. In some embodiments, the RMS period is defined as 250 RMS data points per second (or RMS over 0.004 s). Those of ordinary skill in the art would appreciate that the RMS period can be any RMS period not inconsistent with the objectives of this disclosure, and that the RMS period will be based on the physical properties of sample 70 being tested, the type and sensitivity of microphone 140, the software being used to process the data, the goals of the experiment, etc.

In an embodiment, acoustic apparatus 100 is set up as described in Section III, using microphone 140. An exemplary method of determining cumulative popping signal over time is: placing a sample 70 of a known quantity of gasified crystals on the fritted disc 60; positioning microphone 140 over sample 70 at a known, fixed distance; injecting fluid into wetting cup 1 to wet the fritted disc 60; recording popping sound intensity from gasified crystals dissolving in fluid over a time interval, converting the recorded popping sounds from voltage response units to pressure (Pa) units; subtracting background signal from total signal; and calculating area under curve of the signal by multiplying the Background Corrected Signal by the RMS time step and summing each time step across the full test.

### EXAMPLE 1

### Consumer Preference Studies—Popping Intensity and Duration

A first consumer preference study of 178 participants was conducted to gauge sensory desirability of gasified crystal-containing formulations. Each participant consumed one flavored formulation having gasified crystals with a particle size of 0.5 mm to 1.0 mm each day for three days. The participants ranged in age from 18-65 years old and were 50% male/50% female. Popping intensity and popping duration were two of the physical properties that were evaluated. It was found that 25% of the participants found the popping sensation to be too intense for daily consumption, and 21% of the participants stated that the duration of popping was too long, with approximately 10 seconds of popping being found to be the most desired duration.

A second consumer preference study of 372 participants was conducted using a modified formulation containing half the amount of gasified crystals (to reduce popping duration) and with a more narrow particle size (0.5-0.6 mm) (to reduce popping intensity). Popping intensity and popping duration were two of the physical properties that were evaluated. It was found that 77% of the participants found the popping duration just about right (less than 10 seconds), with only 8% finding the duration of popping to be too long. Additionally, 81% liked the popping intensity.

A third consumer preference study of 58 participants was conducted using the same formulation as the second consumer preference study, except the gasified crystals had a particle size of 0.55 mm to 0.66 mm. Popping intensity and popping duration were two of the physical properties that were evaluated. In this study, participants consumed one flavored formulation having gasified crystals with a particle size of 0.54 mm to 0.66 mm. It was found that only 4% of participants found the popping sensation to be too intense for daily consumption, and no participant found the popping duration to be too long.

Consequently, by reducing the particle size of the gasified crystals to a narrower particle size window that did not have crystals >0.66 mm and/or >0.85 mm, the popping intensity and duration were significantly improved over those found in the first consumer preference study using gasified crystals having particle sizes >0.85 mm.

### EXAMPLE 2

### Consumer Preference Study—Particle Size Ranges and Product Uniformity

When using gasified crystals in nutritional, wellness, or pharmaceutical products, where the gasified crystals are blended with additional ingredients, such as vitamins, minerals, biological actives, API's, sweeteners, and the like, blending uniformity of these ingredients is important to achieve the appropriate dosages and to prevent accumulation and segregation of individual ingredients.

It has been discovered that larger particle sizes of gasified crystals are problematic when blended with other additional ingredients. Specifically, it has been found that gasified crystals having a particle size greater than 0.85 mm fail to blend properly to produce a uniform distribution of the gasified crystals with other ingredients. However, it has been found that gasified crystals having a particle size of 0.85 mm or smaller produce improved uniform distribution of gasified crystals with other ingredients.

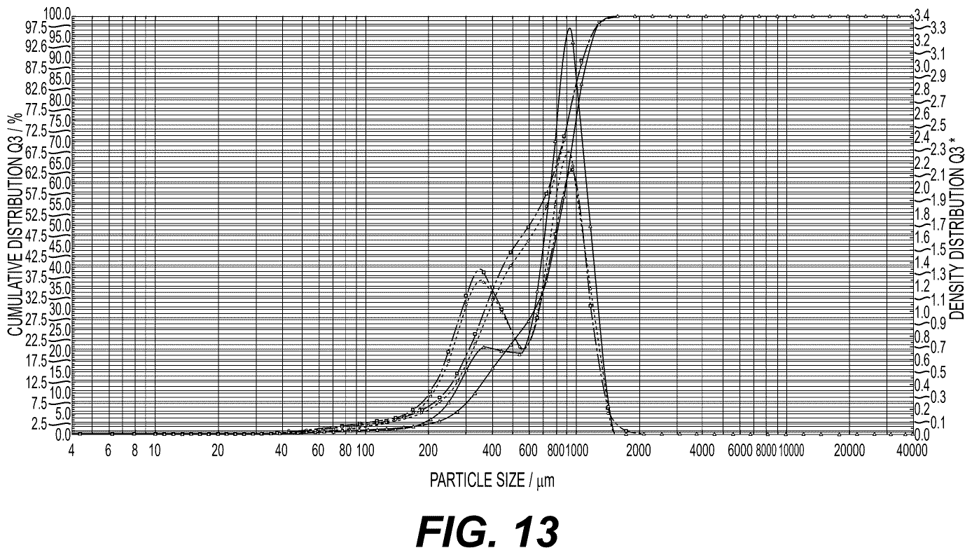

For example, a first lab scale batch was prepared having a serving size proportions of 700 mg of gasified crystals with a size of 0.5 mm to 1.0 mm was blended with 5 mg of zinc, 500 mg of Vitamin C, 0.5 mg of manganese, 2 mg of Vitamin B6, 0.012 mg of vitamin B12, 0.015 mg of vitamin D3, 0.1 mg of folic acid, 100 mg of tapioca maltodextrin, 2500 mg of fructose, and 55 mg of orange flavorant. As shown in FIG. 13, the larger gasified crystals resulted in poor product uniformity in the blend.

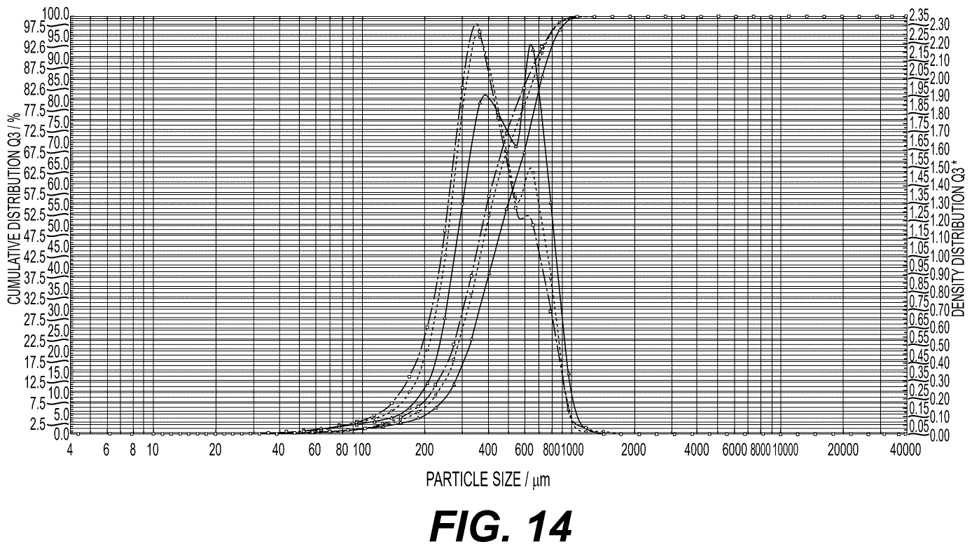

A second lab scale batch was prepared having the same serving size proportions as the first lab scale batch, but with the different being that the 700 mg of gasified crystals had a particle size of 0.54 mm to 0.66 mm. As shown in FIG. 14, the smaller particle size resulted in a dramatic improvement in the blending of the gasified crystals with the additional ingredients compared to the gasified crystals having larger particle sizes.

### EXAMPLE 3

### Measuring Cumulative Popping Signals

To determine cumulative popping signal over time (Paᵣₘₛ*sec) (time domain), a response curve is generated using the acoustic apparatus 100 described in Section III and the methods describe in Section IV herein. Specifically, a fill syringe is attached to the acoustic apparatus and filled with approximately 50 ml of water. The microphone is placed in the interior receiving space of the acoustic apparatus and adjusted to be 51 mm from the top of the fritted disc (145-174 μm) on the wetting cup. To ensure wetting repeatability, a 1″ dosing ring is placed on the fritted disc and a sample of gasified crystals (0.250 g+−0.002 g) is placed evenly in the dosing ring. The water is then injected into wetting cup and the audio recording begins for a predetermined duration (such as 10 minutes) and a known sampling rate (such as 192 ks/s). The quantity of water injected into the wetting cup will depend on the volume of the fluid receiving space in the fluid receiving bowl of the wetting cup. As discussed previously herein, typically the volume of water injected will be slightly in excess of the volume of the fluid receiving space so that some water overflows the fluid receiving bowl into the fluid overflow base.

After recording has finished, each .wav file (raw data from each run) is exported from a microphone input path to a file input path to provide a Voltage RMS response (“Vrms”, 250 times per second) from the file over time. To do this, a High Pass filter (Elliptic 800 Hz) and a Low Pass filter (Butterworth 20 kHz) are applied, 250/s is set as the RMS interval, and all units are set to Vrms. The data is then exported, with each Vrms being converted to pressure (Pa) units by applying the microphone sensitivity from a calibration sheet provided by the manufacturer.

For the microphone in use in this experiment this is 50.79 mV/Pa (Example 0.05079V/(50.79 m V/Pa/1000)=1 Paᵣₘₛ).

The average background noise is then calculated. This is done by averaging the first 5 s of the recording (in Paᵣₘₛ). Background Paᵣₘₛ Average, Background Paᵣₘₛ Standard Deviation, and Background Paᵣₘₛ Average+3*Standard Deviation are then calculated.

Next, the background corrected signal is determined by subtracting the background signal from the total signal. First for each Paᵣₘₛ it is determined whether the signal is greater than Background Average+3*standard deviation. If so then the signal can be differentiated from the background. If the Paᵣₘₛ signal can be differentiated from the background noise then subtract the background to determine the background corrected signal, although it is noted that if the background and signal are not in phase or of the same frequency simple subtraction of the signals is not possible. Calculation of the background correct signal=(Sqrt(TotalSignal²−Background²).

To calculate the area under the curve, the Background Corrected Signal is multiplied by the RMS time step (0.004 s). Each time step is then summed across the full test to get the total area under the curve (Paᵣₘₛ*sec).

### EXAMPLE 4

### Cumulative Popping Signal—Particle Size Ranges

Cumulative popping signals for gasified crystals having different particle size ranges were determined using the method described in EXAMPLE 3. In this experiment, the microphone was a GRAS146AE made by GRAS Sound and Vibration and the Audio Analyzer was Apx517B made by Audio Precision.

Three samples of gasified crystals having different particle size ranges were prepared, with Sample 1 being 0.546 mm to 0.66 mm, Sample 2 being 0.6 mm to 0.85 mm, and Sample 3 being 0.85 mm to 1.00 mm. Nine replicates of Samples 1 and 2 were prepared and tested, and 8 replicates of Sample 3 were prepared and tested. Each sample was approximately 250 mg and the audio recording for each replicate was for 10 minutes. Testing was performed by testing batches of 2 or 3 replicates each day for three days in order to account for some variability caused by ambient conditions.

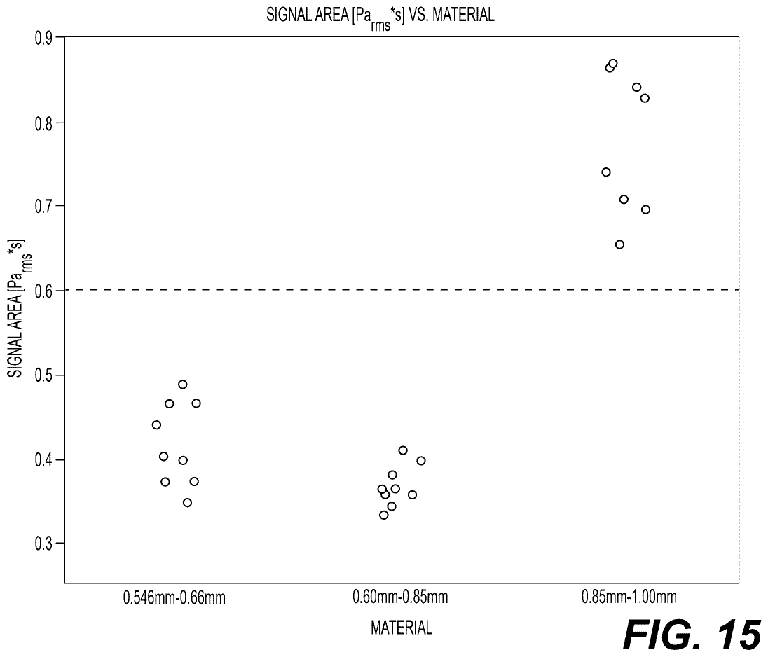

FIG. 15 shows a graph of Signal Area (Paᵣₘₛ*sec) vs. each of Samples 1-3. As shown, for Sample 1 having gasified crystals with a particle size of 0.546 mm to 0.66 mm and Sample 2 having gasified crystals with a particle size of 0.6 mm to 0.85 mm, both showed a cumulative popping signal of 0.6 Paᵣₘₛ*sec or lower over 10 minutes. In contrast, Sample 3 having gasified crystals with a particle size of 0.85 mm to 1.00 mm showed a cumulative popping signal of greater than 0.6 Paᵣₘₛ*sec over 10 minutes.

While not intending to be bound by theory, based on cumulative signal area of 0.6 Paᵣₘₛ*sec or less over 10 minutes (as shown in FIG. 15), it is believed that gasified crystals having a particle size of 0.2 mm to 0.85 mm produce a popping intensity upon dissolution by water that is preferred by consumers. In contrast, due to the jump in the cumulative signal area to greater than 0.6 Paᵣₘₛ*sec over 10 minutes for gasified crystals having a particle size of 0.85 mm to 1 mm (or larger), that the popping intensity upon dissolution by water is not preferred by consumers.

Moreover, based on the poor composition blending uniformity seen for blends with gasified crystals having particle sizes greater than 0.85 mm (FIG. 13), and the improved blending uniformity for blends having gasified crystals with particle sizes less than 0.85 mm (FIG. 14), the smaller gasified crystals show superior physical properties more suited for nutritional, wellness, and pharmaceutical products.

### EXAMPLE 5

### Comparative Testing

Comparative testing of cumulative popping signals for gasified crystals having the different particle size ranges described in EXAMPLE 4 were tested against commercially available Pop Rocks and ICEE popping candies using the method described in EXAMPLE 3. In this experiment, the microphone was a GRAS146AE made by GRAS Sound and Vibration and the Audio Analyzer was Apx517B made by Audio Precision.

Three samples of gasified crystals having different particle size ranges were prepared, with Sample 1 being 0.546 mm to 0.66 mm, Sample 2 being 0.6 mm to 0.85 mm, and Sample 3 being 0.85 mm to 1.00 mm. Additionally, the commercially available Pop Rocks Crackling Candy (Sample 4) and ICEE popping candy (Sample 5) were acquired and prepared. Nine replicates of Samples 1 and 2 were prepared and tested, eight replicates of Sample 3 were prepared and tested, seven replicates of Sample 4 were prepared and tested, and nine replicates of Sample 5 were prepared and tested. In accordance with EXAMPLE 4, each replicate for Samples 1-5 was approximately 250 mg. Testing was performed by testing batches of 2 or 3 replicates each day for three days in order to account for some variability caused by ambient conditions.

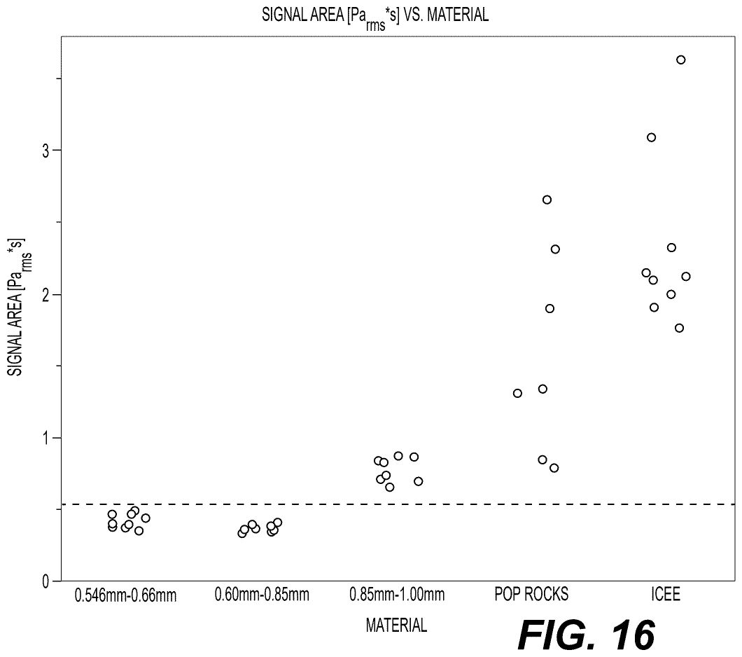

While the methods described in EXAMPLES 3 and 4 were repeated for EXAMPLE 5, there were two notable differences. Firstly, as previously discussed herein, the average particle size in Pop Rocks and ICEE is fairly large, with the larger particle sizes being >1 mm in diameter and often being as large as 4 mm in diameter. This results in a longer period of time in which popping occurs, since the larger crystals take longer to dissolve. To account for this, the length of audio recording for each replicate was increased from 10 minutes to 20 minutes to allow all popping to occur. Secondly, the voltage input range was increased to 2.5V from 0.8V due to a stronger signal from the Pop Rocks and ICEE samples. Neither of these changes was observed to give inconsistent or contradictory results compared to the results of EXAMPLE 4 and FIG. 15, with the data being consistent for Samples 1-3 in both EXAMPLES 4 and 5 and FIGS. 15 and 16.

FIG. 16 shows a graph of Signal Area (Paᵣₘₛ*sec) vs. each of Samples 1-5. As illustrated, for Sample 1 having gasified crystals with a particle size of 0.546 mm to 0.66 mm and Sample 2 having gasified crystals with a particle size of 0.6 mm to 0.85 mm, both showed a cumulative popping signal of 0.6 Paᵣₘₛ*sec or lower over 10 minutes, which is consistent with the results described in EXAMPLE 4 and FIG. 15. For Sample 3 having gasified crystals with a particle size of 0.85 mm to 1.00 mm, the cumulative popping signal was observed to be greater than 0.6 Paᵣₘₛ*sec over 10 minutes, which was also consistent with the results described in EXAMPLE 4 and FIG. 15.

For Sample 4 (Pop Rocks) and Sample 5 (ICEE), both showed a cumulative popping signal of greater than 0.6 Paᵣₘₛ*sec over 20 minutes. As shown in FIG. 16, the cumulative popping signal for each replicate varied wildly, ranging from a cumulative popping signal of approximately 0.8 Paᵣₘₛ*sec up to approximately 3.6+Paᵣₘₛ*sec. The wide variability is believed to be attributed to the larger gasified crystal particle sizes and lack of uniformity in the particle sizes between samples. Additionally, the dramatic increase and variability in popping intensity of the Pop Rocks and ICEE commercial product upon dissolution by water is consistent with the negative consumer feedback.

1. A composition comprising:
gasified crystals having a particle size of 0.2 mm to 0.85 mm, wherein a 250 mg sample of the gasified crystals generates a cumulative popping signal of up to 0.6 Paᵣₘₛ*sec over 10 minutes of dissolution by water.

2. The composition of claim 1, wherein the cumulative popping signal is between 0.2 to 0.6 Paᵣₘₛ*sec.

3. The composition of claim 1, wherein the gasified crystals comprise:
a sugar; and
pressurized gas entrapped in cavities within the gasified crystals.

4. The composition of claim 3, wherein the sugar comprises glucose, fructose, sucrose, lactose, maltose, com syrup, glucose syrup, or any combination thereof.

5. (canceled)

6. The composition of claim 3, wherein the gasified crystals further comprise an acidulant.

7. The composition of claim 6, wherein the acidulant is malic acid.

8. The composition of any claim 3, wherein the gas comprises carbon dioxide, nitrogen, air, a noble gas, or any combination thereof.

9. The composition of claim 3, wherein the pressurized gas has a pressure greater than 1 atmosphere.

10. The composition of claim 1, further comprising one or more vitamins, minerals, biological actives, active pharmaceutical ingredients, or any combination thereof.

11. The composition of claim 10, wherein vitamins comprise vitamin A, a vitamin B (including B3, B6, B9, B12, and biotin), vitamin C, vitamin D, vitamin E, vitamin K, thiamin, riboflavin, niacin, folate, pantothenic acid, pharmaceutically acceptable salts thereof, or combinations thereof.

12. The composition of claim 10, wherein minerals comprise iron, phosphorus, iodine, copper, chromium, molybdenum, chloride, sodium, magnesium, calcium, zinc, selenium, manganese, potassium, choline, silicon, pharmaceutically acceptable salts thereof, or any combination thereof.

13. The composition of claim 10, wherein the biological active comprises lycopene, lutein, L-threonine, coenzyme Q-10, phytosterols, hyaluronic acid, cognizing citicoline, icotinamide, Andrographis, or any combination thereof.

14. The composition of claim 10, wherein the active pharmaceutical ingredient comprises a pain or inflammation reducer, an antihistamine, a decongestant, a cough suppressant, a biological active, a sleep aid, a stress-reducing aid, an energy and cognition aid, an Ayurveda supplement, an immunity supplement, a digestive aid, a joint supplement, a cannabinoid, a systemic natural, a nutritional agent, pharmaceutically acceptable salts thereof, or combinations thereof.

15. The composition according to claim 1, further comprising a sweetener.

16. The composition of claim 15, wherein the sweetener comprises glucose, fructose, sucrose, lactose, maltose, corn syrup, or any combination thereof.

17. The composition of claim 1, further comprising a diluent, a flavorant, a colorant, a preservative, or any combination thereof.

18. (canceled)

19. A composition comprising:
gasified crystals having a particle size of 0.25 to 0.85 mm, the gasified crystals comprising sucrose, glucose syrup, and a pressurized gas entrapped in cavities within the gasified crystals;
wherein a 250 mg sample of the gasified crystals generates a popping signal of 0.2 to 0.6 Paᵣₘₛ*sec over 10 minutes of dissolution by water.

20. The composition of claim 19, wherein the gas is carbon dioxide pressurized at greater than 1 atmosphere.

21. The composition of claim 19, further comprising one or more vitamins, minerals, biological actives, active pharmaceutical ingredients, or any combination thereof.

22. The composition of claim 19, further comprising an acidulent.

23. (canceled)
